(19) United States
(12) Patent Application Publication
(10) Pub. No.: US 2020/0048162 A1
(43) Pub. Date: Feb. 13, 2020

(54) PREPARATION METHOD FOR CONJUGATED DIENE COMPOUND

(71) Applicant: DALIAN UNIVERSITY OF TECHNOLOGY, Dalian City, Liaoning Province (CN)

(72) Inventors: Ming BAO, Dalian City, Liaoning Province (CN); Hongyu GUO, Dalian City, Liaoning Province (CN); Sheng ZHANG, Dalian City, Liaoning Province (CN)

(21) Appl. No.: 16/342,894

(22) PCT Filed: Jun. 7, 2018
(86) PCT No.: PCT/CN2018/090254

§ 371 Date: Apr. 17, 2019

(30) Foreign Application Priority Data
Sep. 15, 2017 (CN) 201710830258.8

(51) Int. Cl. C07C 2/40

(52) U.S. Cl. CPC C07C 2/406; C07C 2527/185; C07C 15/50; C07C 2531/22; C07C 2531/28; C07C 2531/24

(57) ABSTRACT

The current invention belongs to the technical fields of fine chemicals and related chemistry, and provides a preparation method for butadiene derivatives. Arylacetylenes and derivatives using as raw materials react in an anhydrous organic solvent in the presence of a metal catalyst and an additive, and are converted into 2,3-disubstituted-1,3-butadiene derivatives. The current invention has some beneficial characteristics such as cheap and readily available raw material, mild reaction conditions, environmentally friendly property and possibility of realizing industrialization, and obtains the 1,3-butadiene derivatives in high yields. The 1,3-butadiene derivatives synthesized by this method can be further functionalized into various compounds which have potential applications in development and research of natural products, functional materials and fine chemicals.

### TECHNICAL FIELD

The current invention belongs to the technical fields of fine chemicals and related chemistry, and provides a preparation method for 1,3-butadiene derivatives.

### BACKGROUND

Conjugated diene derivative is an important part of many natural product molecules, is a kind of important intermediate in organic synthesis, and is widely used in Diels-Alder reaction, electrocyclic reaction and Ziegler-Natta polymerization [Mundal D. A., Lutz K. E., Thomson R. J., Org. Lett., 2009, 11, 465-468]. Therefore, the development of high-efficiently and high-selectively synthetic methods of conjugated dienes, especially 1,3-butadiene derivatives, is significant and has application value. At present, the main methods for synthesis of 1,3-butadiene derivatives are: Wittig reaction, transition metal-catalyzed cross-coupling reaction of alkenyl halides, and Pd-catalyzed oxidative coupling reaction of N-tosylhydarazones.

Conjugated dienes are obtained by the reaction of aldehydes with phosphorus ylides in the Wittig reaction. The reaction requires higher requirements for operation and conditions. Firstly, n-BuLi or t-BuLi is used to convert phosphine salt to obtain phosphorus ylides, and then aldehydes are added to the reaction system to provide conjugated dienes. Moreover, the raw material of the reaction needs to be excessive, so it is limited in industrial application [Maryanoff B. E., Reitz A. B. Chem. Rev., 1989, 89, 863-927]. Since the 1970s, the transition metal-catalyzed cross-coupling reaction has attracted extensive attention due to its high efficiency and has become an important method for constructing carbon-carbon bonds. However, the method requires not only higher reaction temperature, but also pre-functionalization of reaction substrates [Lee, P. H., Seomoon D., Lee K. Org. Lett., 2005, 7, 343-345]. Therefore, how to improve the atomic economy, reduce the temperature of the system and use cheaper and readily available substrates are the current challenges.

### SUMMARY

The current invention provides a novel preparation method for 1,3-butadiene derivatives. Mild and environmentally friendly reaction conditions, experimental simplicity, cheaper and readily available in raw materials and high yield are the useful features of current catalytic method.

The current invention adopts the following technical solution:

A preparation method for conjugated diene compounds is provided. arylacetylene derivatives using as raw materials react in an anhydrous organic solvent at 20° C.−80° C. in the presence of a metal catalyst and an additive for 12-24 hours, and are converted into 1,3-butadiene derivatives, with a synthetic route as follows:

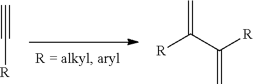

R is selected from alkyl and aryl;

a molar ratio of the arylacetylene derivative to the metal catalyst is 1:0.02 to 1:0.1;

a molar ratio of the arylacetylene derivative to the additive is 1:0.1 to 1:2; and

the molar concentration of the arylacetylene derivative is 0.01 mmol/mL to 2 mmol/mL.

The solvent comprises tetrahydrofuran, 1,2-dimethoxyethane, chloroform, dichloromethane, diethyl ether, dimethyl sulfoxide, carbon tetrachloride, acetone, toluene, 1,4-dioxane, N,N-dimethylformamide and hexane, and preferably dichloromethane, tetrahydrofuran and acetone.

The catalyst comprises Pd₂dba₃, Pd(PPh₃)₄, Pd(PPh₃)₂Cl₂, Pd(OAc)₂, Pd(TFA)₂, PdCl₂, Pd(CH₃CN)₂Cl₂ and Pd(acac)₂, and preferably Pd(PPh₃)₄, Pd₂dba₃, Pd(PPh₃)₂Cl₂, Pd(OAc)₂ and Pd(acac)₂.

Ligands comprise PPh₃, tri(p-tolyl)phosphine, tri(2-furyl)phosphine, PCy₃, Ph₂P^{t}Bu, P^{t}Bu₃, PEt₃, tri(o-tolyl)phosphine, Me₂PPh and PⁿBu; and preferably PPh₃, tri(p-tolyl)phosphine and Ph₂P^{t}Bu. A molar ratio of the metal catalysts to the ligands is 1:2 to 1:4.

The additive 1 comprises iron powder, manganese powder, magnesium powder and zinc powder, and preferably iron powder, zinc powder and manganese powder.

The additive 2 comprises toluene-p-sulfonic acid, 2,6-pyridinedicarboxylic acid, trifluoromethanesulfonic acid, trimethylacetic acid, salicylic acid, trifluoroacetic acid, methanesulfonic acid, 2-ethylhexanoic acid, m-nitrobenzoic acid and cinnamic acid, and preferably toluene-p-sulfonic acid, trifluoromethanesulfonic acid, trimethylacetic acid, trifluoroacetic acid and methanesulfonic acid.

Separation methods comprise recrystallization and column chromatography. The recrystallization method uses solvents including benzene, alcohol, petroleum ether, acetonitrile, tetrahydrofuran, chloroform, hexane, acetone, ethyl acetate and dichloromethane. When the column chromatography method is used to separate products, silica gel or alumina can be used as a stationary phase and an eluent is generally a mixture of polar and nonpolar solvents such as ethyl acetate-petroleum ether, ethyl acetate-hexane, dichloromethane-petroleum ether and methanol-petroleum ether.

The current invention has some beneficial characteristics such as cheap and readily available raw material, mild reaction conditions, environmentally friendly property and possibility of realizing industrialization, and obtains the 1,3-butadiene derivatives in high yields. The 1,3-butadiene derivatives synthesized by this method can be further functionalized into various compounds which have potential applications in development and research of natural products, functional materials and fine chemicals.

### DESCRIPTION OF DRAWINGS

FIG. 1 is a ¹H NMR of buta-1,3-diene-2,3-diyldibenzene in example 1.

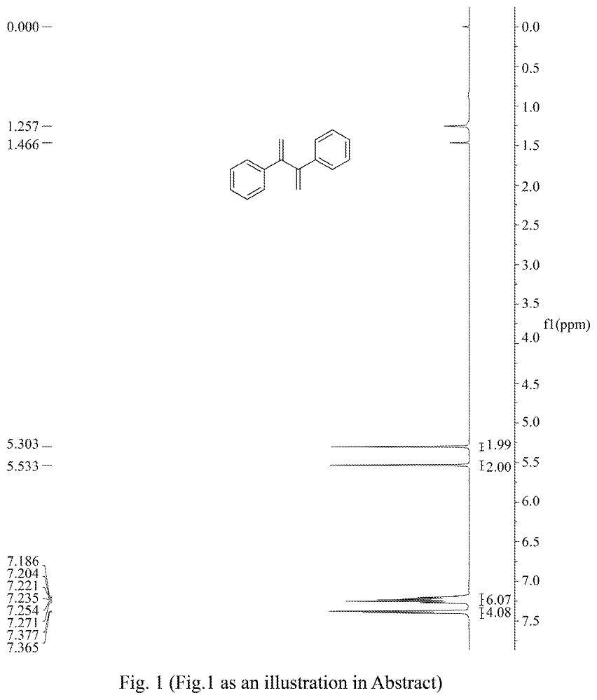

FIG. 2 is a ¹³C NMR of buta-1,3-diene-2,3-diyldibenzene in example 1.

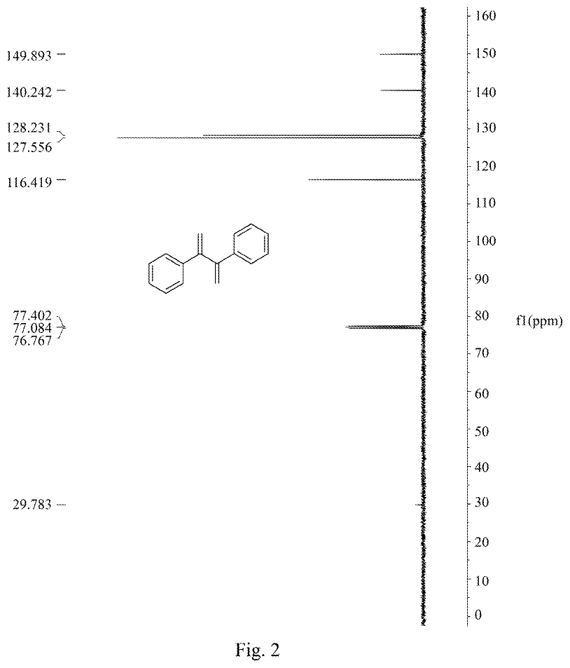

FIG. 3 is a ¹H NMR of 4,4′-(buta-1,3-diene-2,3-diyl)bis(methylbenzene) in example 2.

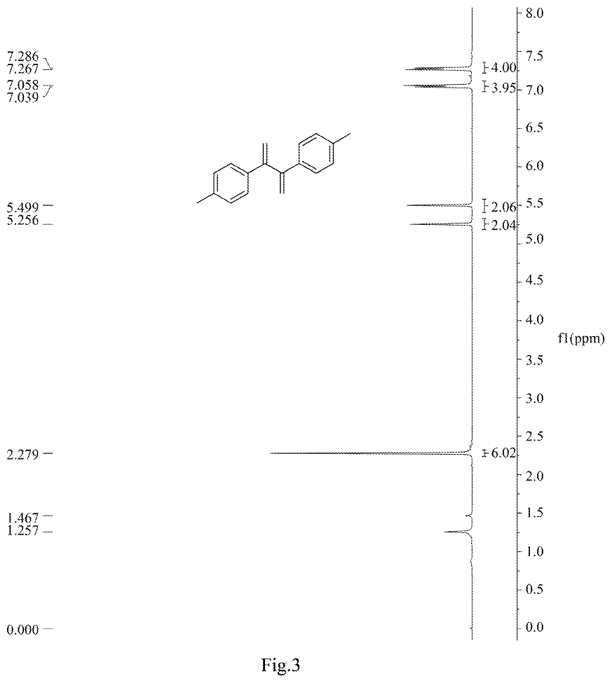

FIG. 4 is a ¹³C NMR of 4,4′-(buta-1,3-diene-2,3-diyl)bis(methylbenzene) in example 2.

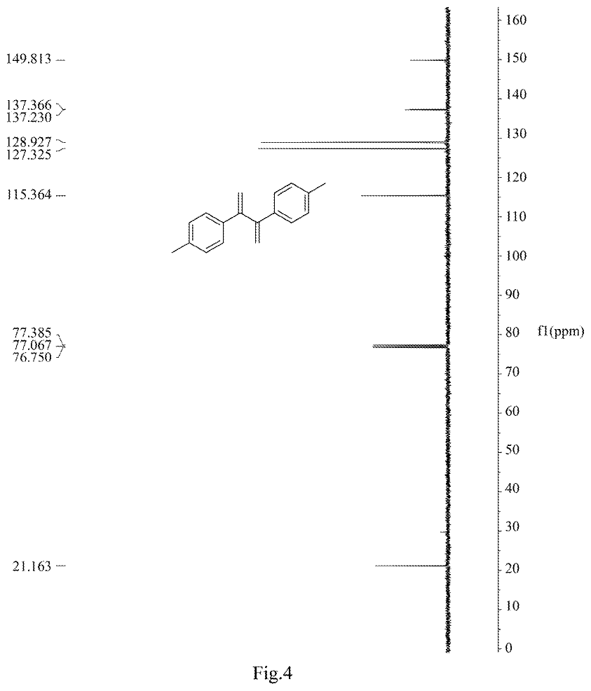

FIG. 5 is a ¹H NMR of 4,4′-(buta-1,3-diene-2,3-diyl)bis(methoxybenzene) in example 3.

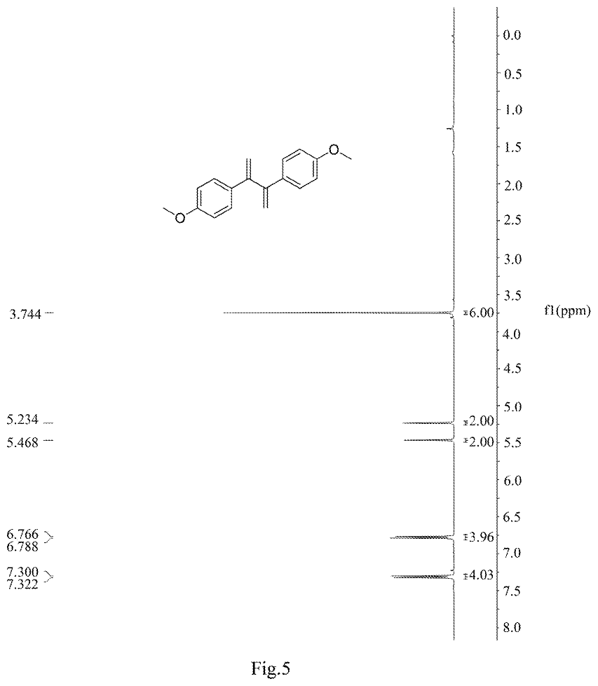

FIG. 6 is a ¹³C NMR of 4,4′-(buta-1,3-diene-2,3-diyl)bis(methoxybenzene) in example 3.

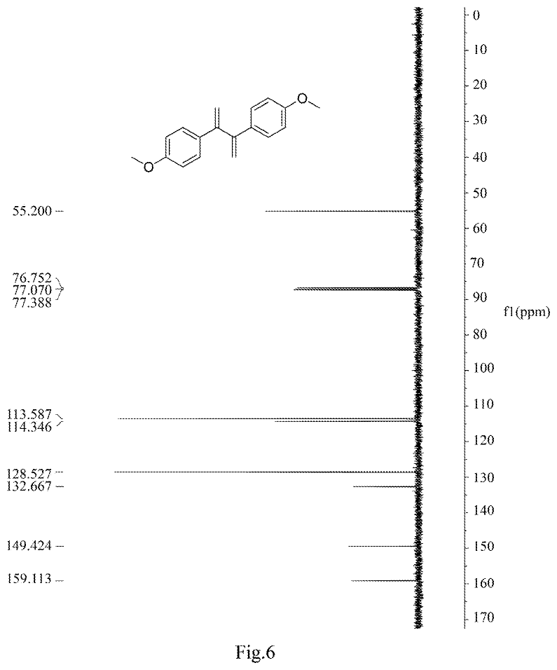

FIG. 7 is a ¹H NMR of 4,4′-(buta-1,3-diene-2,3-diyl)bis(fluorobenzene) in example 4.

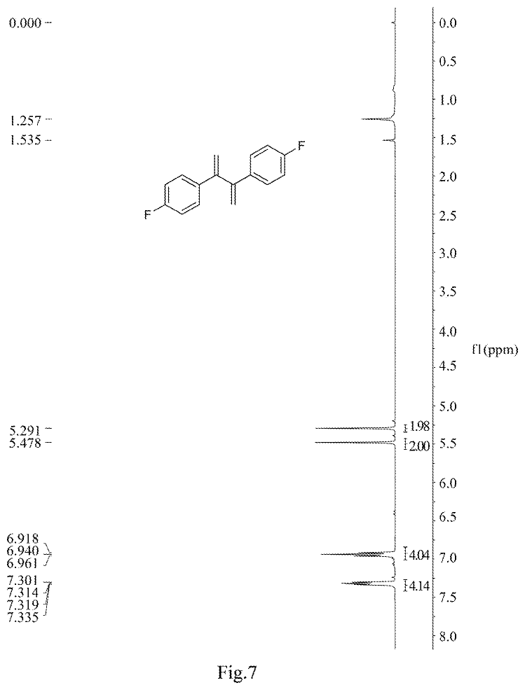

FIG. 8 is a ¹³C NMR of 4,4′-(buta-1,3-diene-2,3-diyl)bis(fluorobenzene) in example 4.

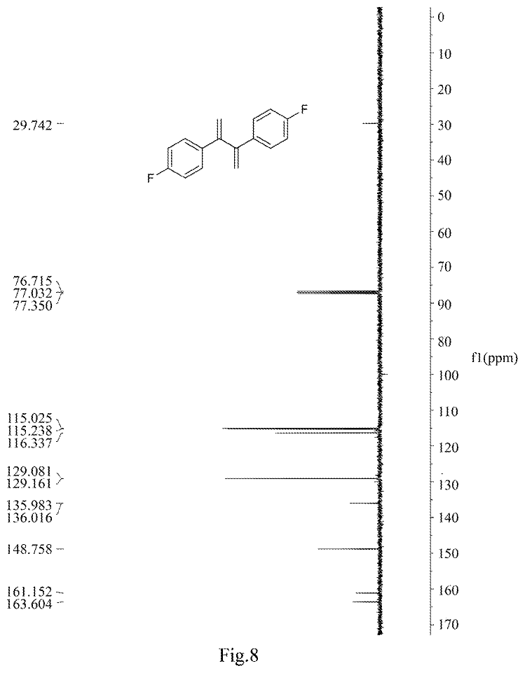

FIG. 9 is a ¹H NMR of 4,4′-(buta-1,3-diene-2,3-diyl)bis(bromobenzene) in example 5.

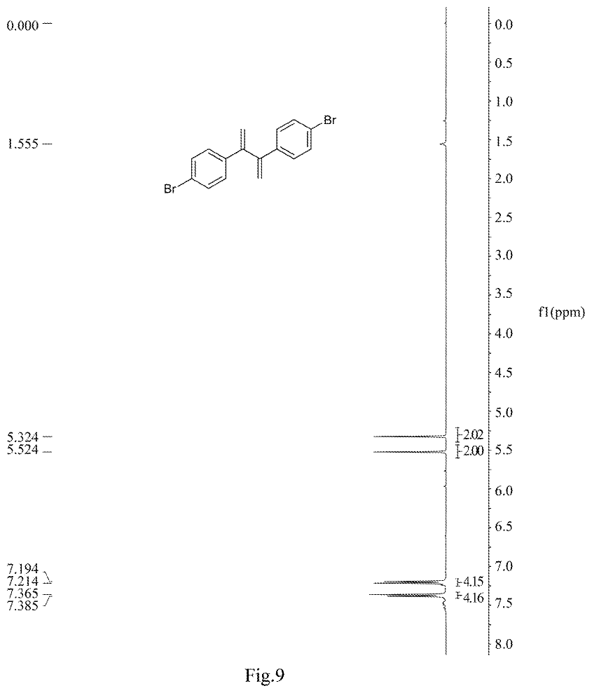

FIG. 10 is a ¹³C NMR of 4,4′-(buta-1,3-diene-2,3-diyl)bis(bromobenzene) in example 5.

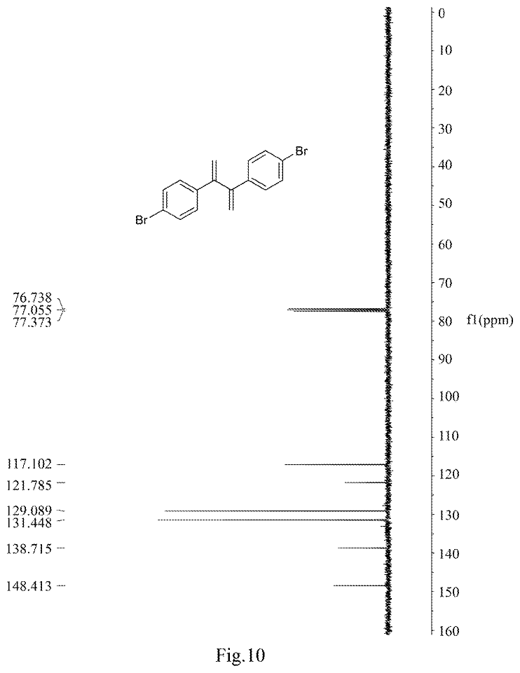

FIG. 11 is a ¹H NMR of 1,1′-(buta-1,3-diene-2,3-diylbis(4,1-phenylene))bis(ethan-1-one) in example 6.

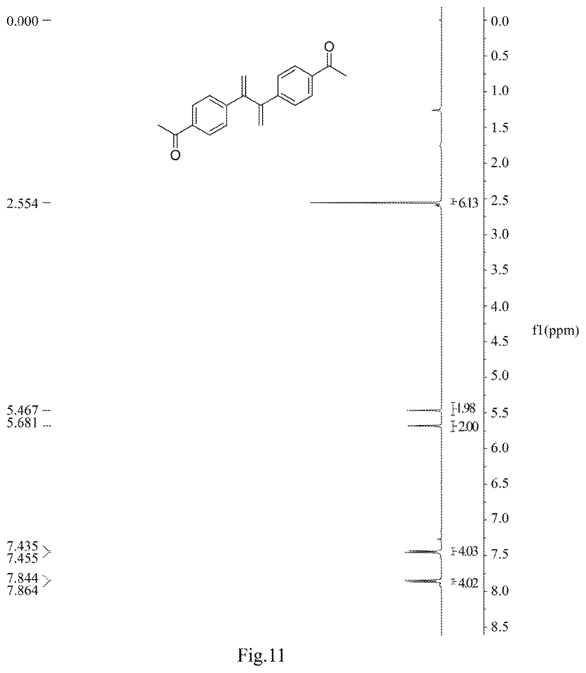

FIG. 12 is a ¹³C NMR of 1,1′-(buta-1,3-diene-2,3-diylbis(4,1-phenylene))bis(ethan-1-one) in example 6.

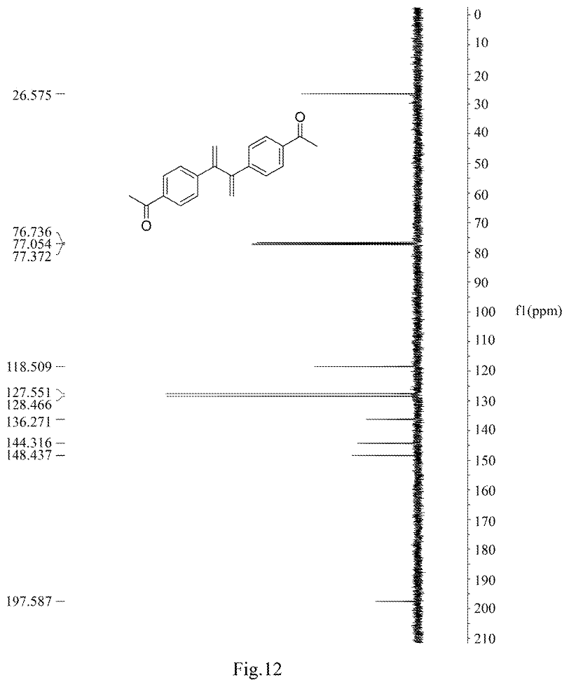

FIG. 13 is a ¹H NMR of 2,2′-(buta-1,3-diene-2,3-diyl)dithiophene in example 7.

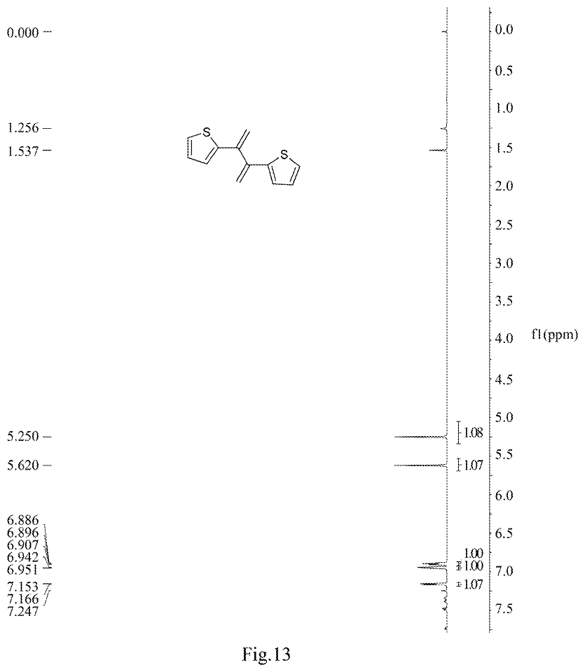

FIG. 14 is a ¹³C NMR of 2,2′-(buta-1,3-diene-2,3-diyl)dithiophene in example 7.

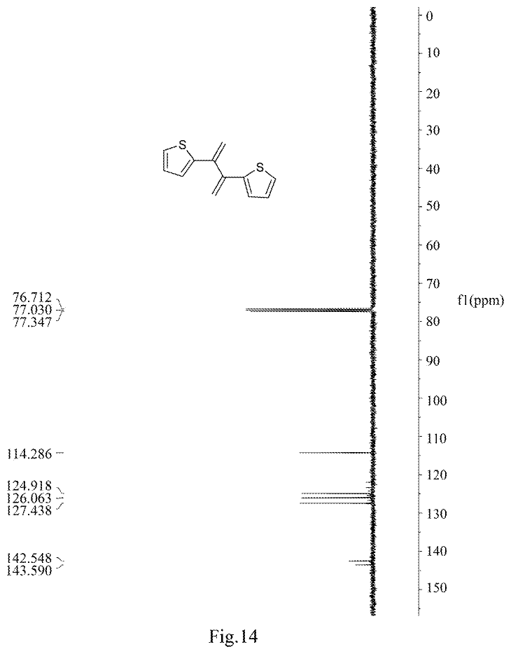

FIG. 15 is a ¹HNMR of 7,8-dimethylenetetradecane in example 8.

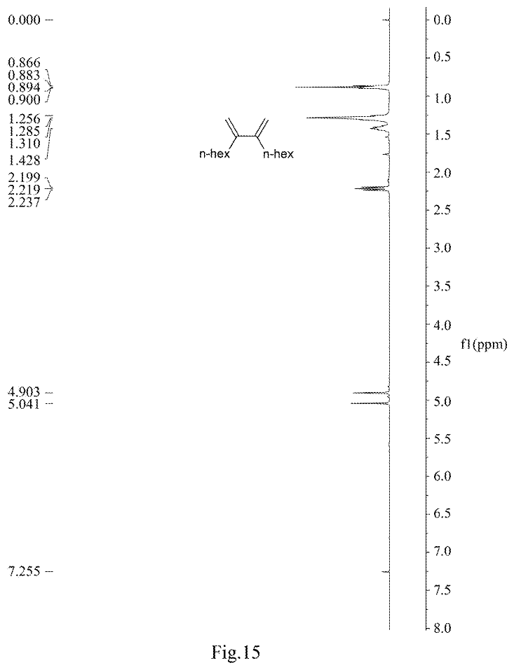

FIG. 16 is a ¹³C NMR of 7,8-dimethylenetetradecane in example 8.

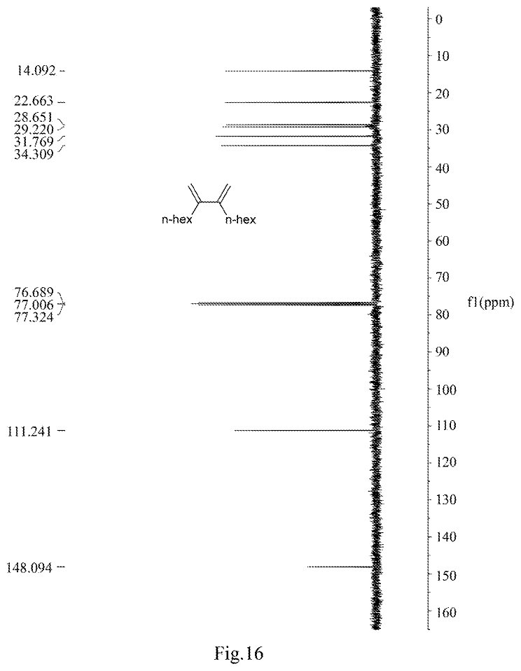

### DETAILED DESCRIPTION

The preparation method for the 1,3-butadiene derivatives in the current invention has the advantages of low cost of raw material, fewer reaction steps, mild reaction conditions, environmentally friendly property, convenient operation and high reaction yield.

The current invention is further described below in combination with the specific examples. The examples are only used for illustrating the current invention, not used for limiting the scope of the current invention. Simple replacement or improvement made to the current invention by those skilled in the art belongs to the technical solution protected by the current invention.

### Example 1: Synthesis of buta-1,3-diene-2,3-diyldibenzene

In a 25 mL reactor toluene-p-sulfonic acid (0.029 g, 0.15 mmol), trimethylacetic acid (0.087 g, 0.85 mmol), zinc powder (0.033 g, 0.5 mmol) and Pd(PPh₃)₄ (0.023 g, 0.02 mmol) are added; nitrogen is exchanged for three times; then 3 mL of anhydrous dichloromethane is added; arylacetylenes (0.051 g, 0.5 mmol) is added while stirred; and the above mixtures are stirred for 24 h at 25° C. Column chromatography separation (silica gel with 200-300 meshes; eluent, petroleum ether) is conducted to obtain 0.038 g of buta-1,3-diene-2,3-diyldibenzene with a yield of 73%.

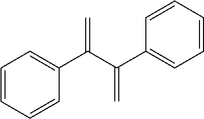 [SMILES: C=C(C(=C)c1ccccc1)c1ccccc1]

### buta-1,3-diene-2,3-diyldibenzene

Clear crystal; ¹H NMR (CDCl₃, 400 MHz) δ 7.38-7.40 (m, 4H), 7.19-7.27 (m, 6H), 5.53 (s, 2H), 5.30 (s, 2H); ¹³C NMR (CDCl₃, 100 MHz) δ 149.9, 140.2, 128.2, 127.6, 116.4 ppm; MS (D) m/z=207, 206, 191, 178, 128, 115, 91.

### Example 2: Synthesis of 4,4′-(buta-1,3-diene-2,3-diyl)bis(methylbenzene)

Operation is the same as that in example 1. The 1-ethynyl-4-methylbenzene reacts to produce 0.049 g of 4,4′-(buta-1,3-diene-2,3-diyl)bis(methylbenzene) with a yield of 83%.

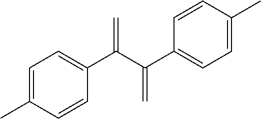

### 4,4′-(buta-1,3-diene-2,3-diyl)bis(methylbenzene)

White solid; ¹H NMR (CDCl₃, 400 MHz) δ 7.28 (d, J=8.0 Hz, 4H), 7.05 (d, J=8.0 Hz, 4H), 5.50 (s, 2H), 5.26 (s, 2H), 2.28 (s, 6H); ¹³C NMR (CDCl₃, 100 MHz) δ149.8, 137.4, 137.2, 128.9, 127.3, 115.4, 21.2 ppm; MS (D) m/z=235, 234, 219, 204, 128, 115, 91

### Example 3: Synthesis of 4,4′-(buta-1,3-diene-2,3-diyl)bis(methoxybenzene)

Operation is the same as that in example 1. The 4-methoxyl arylacetylene reacts to produce 0.060 g of 4,4′-(buta-1,3-diene-2,3-diyl)bis(methoxybenzene) with a yield of 90%.

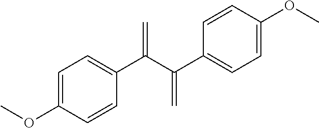

### 4,4′-(buta-1,3-diene-2,3-diyl)bis(methoxybenzene)

White solid; ¹H NMR (CDCl₃, 400 MHz) δ 7.31 (d, J=8.0 Hz, 4H), 6.78 (d, J=8.0 Hz, 4H), 5.47 (s, 2H), 5.23 (s, 2H), 3.74 (s, 6H); ¹³C NMR (CDCl₃, 100 MHz) δ159.1, 149.4, 132.7, 128.5, 114.3, 113.6, 55.2 ppm; MS (EI) m/z=267, 266, 251, 235, 121

### Example 4: Synthesis of 4,4′-(buta-1,3-diene-2,3-diyl)bis(fluorobenzene)

In a 25 mL reactor trifluoromethanesulfonic acid (0.0225 g, 0.15 mmol), trimethylacetic acid (0.087 g, 0.85 mmol), iron powder (0.028 g, 0.5 mmol), Pd₂dba₃ (0.092 g, 0.01 mmol) and PPh₃ (0.011 g, 0.04 mmol) are added; nitrogen is exchanged for three times; then 3 mL of anhydrous acetone is added; 1-ethynyl-4-fluorobenzene (0.061 g, 0.5 mmol) is added while stirred; and the above mixtures are stirred for 24 h at 35° C. Column chromatography separation (silica gel with 200-300 meshes; eluent, petroleum ether) is conducted to obtain 0.045 g of 4,4′-(buta-1,3-diene-2,3-diyl)bis(fluorobenzene) with a yield of 73%.

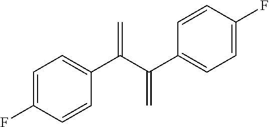 [SMILES: C=C(C(=C)c1ccc(F)cc1)c1ccc(F)cc1]

### 4,4′-(buta-1,3-diene-2,3-diyl)bis(fluorobenzene)

Colorless oil; ¹H NMR (CDCl₃, 400 MHz) δ 7.30-7.34 (m, 4H), 6.92-6.96 (m, 4H), 5.48 (s, 2H), 5.29 (s, 2H); ¹³C NMR (CDCl₃, 100 MHz) δ 163.6, 161.2, 148.8, 136.0 (d, J=3.3 Hz), 129.1 (d, J=8 Hz), 116.3, 115.2, 115.0 ppm; HRMS (EI) m/z calcd. For C₁₆H₁₂F₂: 242.0907; found: 242.0911.

### Example 5: Synthesis of 4,4′-(buta-1,3-diene-2,3-diyl)bis(bromobenzene)

In a 25 mL reactor trifluoromethanesulfonic acid (0.023 g, 0.15 mmol), trimethylacetic acid (0.087 g, 0.85 mmol), manganese powder (0.027 g, 0 5 mmol), Pd(PPh₃)₂Cl₂ (0.014 g, 0.02 mmol) and PPh₃ (0.011 g, 0.04 mmol) are added; nitrogen is exchanged for three times; then 3 mL of anhydrous acetone is added; 1-ethynyl-4-bromobenzene (0.091 g, 0.5 mmol) is added while stirred; and the above mixtures are stirred for 24 h at 20° C. Column chromatography separation (silica gel with 200-300 meshes; eluent, petroleum ether) is conducted to obtain 0.070 g of 4,4′-(buta-1,3-diene-2,3-diyl)bis(bromobenzene) with a yield of 77%.

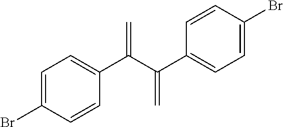 [SMILES: C=C(C(=C)c1ccc(Br)cc1)c1ccc(Br)cc1]

### 4,4′-(buta-1,3-diene-2,3-diyl)bis(bromobenzene)

Yellow crystal; ¹H NMR (CDCl₃, 400 MHz) δ 7.38 (d, J=8 Hz, 4H), 7.20 (d, J=8 Hz, 4H), 5.52 (s, 2H), 5.32 (s, 2H); ¹³C NMR (CDCl₃, 100 MHz) δ 148.4, 138.7, 131.4, 129.1, 121.8, 117.1 ppm; MS (EI) m/z=366, 364, 362, 338, 336, 334, 283, 204, 101

### Example 6: Synthesis of 1,1′-(buta-1,3-diene-2,3-diylbis(4,1-phenylene))bis(ethan-1-one)

In a 25 mL reactor methanesulfonic acid (0.014 g, 0.15 mmol), trimethylacetic acid (0.087 g, 0.85 mmol), manganese powder (0.027 g, 0.5 mmol), tri(2-furyl)phosphine (0.009 g, 0.04 mmol) and Pd(OAc)₂ (0.005 g, 0.02 mmol) are added; nitrogen is exchanged for three times; then 3 mL of anhydrous tetrahydrofuran is added; 1-(4-ethynylphenyl)ethan-1-one (0.072 g, 0.5 mmol) is added while stirred; and the above mixtures are stirred for 24 h at 40° C. Column chromatography separation (silica gel with 200-300 meshes; eluent, petroleum ether) is conducted to obtain 0.033 g of 1,1′-(buta-1,3-diene-2,3-diylbis(4,1-phenylene))bis(ethan-1-one) with a yield of 62%.

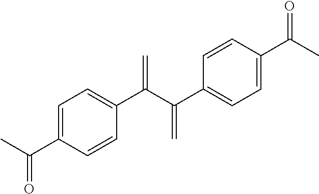 [SMILES: C=C(C(=C)c1ccc(C(C)=O)cc1)c1ccc(C(C)=O)cc1]

### 1,1′-(buta-1,3-diene-2,3-diylbis(4,1-phenylene))bis(ethan-1-one)

White solid; ¹H NMR (CDCl₃, 400 MHz) δ 7.85 (d, J=8 Hz, 2H), 7.45 (d, J=8 Hz, 2H), 5.68 (s, 2H), 5.47 (s, 2H); ¹³C NMR (CDCl₃, 100 MHz) δ 197.6, 148.4, 144.3, 136.3, 128.5, 127.6, 118.5, 26.6 ppm; MS: m/z=291 [M+H⁺]

### Example 7: Synthesis of 2,2′-(buta-1,3-diene-2,3-diyl)dithiophene

In a 25 mL reactor methanesulfonic acid (0.014 g, 0.15 mmol), trifluoroacetic acid (0.097 g, 0.85 mmol), manganese powder (0.027 g, 0.5 mmol), Pd(OAc)₂ (0.005 g, 0.02 mmol) and tri(p-tolyl)phosphine (0.012 g, 0.04 mmol) are added; nitrogen is exchanged for three times; then 3 mL of anhydrous tetrahydrofuran is added; 2-ethynylthiophene (0.0541 g, 0.5 mmol) is added while stirred; and the above mixtures are stirred for 24 h at 50° C. Column chromatography separation (silica gel with 200-300 meshes; eluent, petroleum ether) is conducted to obtain 0.034 g of 2,2′-(buta-1,3-diene-2,3-diyl)dithiophene with a yield of 62%.

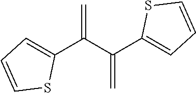

### 2,2′-(buta-1,3-diene-2,3-diyl)dithiophene

Colorless oil; ¹H NMR (CDCl₃, 400 MHz) δ 7.16-7.18 (m, 2H), 6.95-6.96 (m, 2H), 6.90-6.92 (in, 2H), 5.63 (s, 2H), 5.26 (s, 2H); ¹³C NMR (CDCl₃, 100 MHz) δ 143.6, 142.5, 127.4, 126.1, 124.9, 114.3 ppm; HRMS (EI): m/z calcd. For C₁₂H₁₀S₂: 218.0224; found: 218.0227.

### Example 8: synthesis of 7,8-dimethylenetetradecane

In a 25 mL reactor toluene-p-sulfonic acid (0.029 g, 0.15 mmol), trifluoroacetic acid (0.097 g, 0.85 mmol), Zinc powder (0.033 g, 0.5 mmol), PPh₃ (0.011 g, 0.04 mmol) and Pd(acac)₂ (0.006 g, 0.02 mmol) are added; nitrogen is exchanged for three times; then 3 mL of anhydrous dichloromethane is added; oct-1-yne (0.055 g, 0.5 mmol) is added while stirred; and the above mixtures are stirred for 24 h at 25° C. Column chromatography separation (silica gel with 200-300 meshes; eluent, petroleum ether) is conducted to obtain 0.043 g of 7,8-dimethylenetetradecane with a yield of 77%.

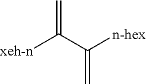

### 7,8-dimethylenetetradecane

Colorless oil; ¹H NMR (400 MHz, CDCl₃): δ5.04 (s, 2H), 4.90 (s, 2H), 2.22 (dd, J=7.2, 7.2 Hz, 4H), 1.44-1.39 (m, 4H), 1.32-1.25 (m, 12H), 0.90-0.86 (t, J=6.8 Hz, 6H) ppm. ¹³C NMR (100 MHz, CDCl₃): δ148.1, 111.2, 34.3, 31.8, 29.2, 0.7, 22.7, 14.1 ppm.

1. A preparation method for conjugated diene compounds, wherein arylacetylene derivatives as raw materials reacting in an anhydrous organic solvent at 20° C.−80° C. in the presence of a metal catalyst and an additive for 12-24 hours, and being converted into 1,3-butadiene derivatives, with a synthetic route as follows: 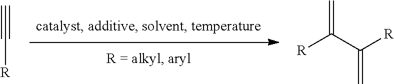
R is selected from alkyl and aryl;
a molar ratio of the arylacetylene derivative to the metal catalyst is 1:0.02 to 1:0.1;
a molar ratio of the arylacetylene derivative to the additive is 1:0.1 to 1:2; and
the molar concentration of the arylacetylene derivative is 0.01 mmol/mL to 2 mmol/mL.

2. The preparation method according to claim 1, wherein the anhydrous organic solvent comprises tetrahydrofuran, 1,2-dimethoxyethane, chloroform, dichloromethane, diethyl ether, dimethyl sulfoxide, carbon tetrachloride, acetone, toluene, 1,4-dioxane, N,N-dimethylformamide and hexane.

3. The preparation method according to claim 1, wherein the catalyst comprises Pd₂dba₃, Pd(PPh₃)₄, Pd(PPh₃)₂Cl₂, Pd(OAc)₂, Pd(TFA)₂, PdCl₂, Pd(CH₃CN)₂Cl₂ and Pd(acac)₂; ligands comprise PPh₃, tri(p-tolyl)phosphine, tri(2-furyl)phosphine, PCy₃, Ph₂P^{t}Bu, P^{t}Bu₃, PEt₃, tri(o-tolyl)phosphine, Me₂PPh and PⁿBu; and a molar ratio of the metal catalysts to the ligands is 1:2 to 1:4.

4. The preparation method according to claim 1, wherein the additive comprises additive 1 and additive 2; the additive 1 comprises iron powder, manganese powder, magnesium powder and zinc powder; and the additive 2 comprises toluene-p-sulfonic acid, 2,6-pyridinedicarboxylic acid, trifluoromethanesulfonic acid, trimethylacetic acid, salicylic acid, trifluoroacetic acid, methanesulfonic acid, 2-ethylhexanoic acid, m-nitrobenzoic acid and cinnamic acid.

5. The preparation method according to claim 3, wherein the additive comprises additive 1 and additive 2; the additive 1 comprises iron powder, manganese powder, magnesium powder and zinc powder; and the additive 2 comprises toluene-p-sulfonic acid, 2,6-pyridinedicarboxylic acid, trifluoromethanesulfonic acid, trimethylacetic acid, salicylic acid, trifluoroacetic acid, methanesulfonic acid, 2-ethylhexanoic acid, m-nitrobenzoic acid and cinnamic acid.
